(19) United States
(12) Patent Application Publication
(10) Pub. No.: US 2019/0369096 A1
(43) Pub. Date: Dec. 5, 2019

(54) VALIDATION OF NEOEPITOPE-BASED TREATMENT

(71) Applicants: NANTBIO, INC., Culver City, CA (US); NANT HOLDINGS IP, LLC, Culver City, CA (US)

(72) Inventors: Kayvan NIAZI, Culver City, CA (US); Shahrooz RABIZADEH, Culver City, CA (US); Nicholas J. WITCHEY, Culver City, CA (US)

(21) Appl. No.: 16/477,505

(22) PCT Filed: Jan. 12, 2018
(86) PCT No.: PCT/US18/13642

§ 371 Date: Jul. 11, 2019

Related U.S. Application Data
(60) Provisional application No. 62/446,191, filed on Jan. 13, 2017.

(51) Int. Cl. G01N 33/574; G01N 33/68

(52) U.S. Cl. CPC G01N 33/574; G01N 2800/52; G01N 33/6878; G01N 33/6863

(57) ABSTRACT

Contemplated systems and methods verify a patient's likely immune response to a neoepitope-based treatment by (a) assessing whether or not the patient's immune system is ready to mount an immune response, (b) determining prior response by patient immune-competent cells, and (c) determining the capability for patient immune-competent cells to respond to a future immune stimulus.

This application claims priority to U.S. provisional application with the Ser. No. 62/446,191, filed Jan. 13, 2017, and which is incorporated by reference herein.

### FIELD OF THE INVENTION

The field of the invention is cancer therapy, especially as it relates to validation of cancer therapy treatments with respect to the immune system of a patient diagnosed with cancer.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Despite recent advances in the diagnosis and treatment of cancer, many types of cancers remain difficult to cure or drive into long term remission. Currently, one promising avenue of research is the individualized therapy of cancers. The concept of individualized therapy is based on the observation that each cancer, in each individual patient or subject, is in some ways unique. Thus, if the therapy is tailored to address or target the genetic elements and/or regulatory cellular pathway(s) altered or mis-regulated in a specific tumor, the prospects of a successful treatment against the tumor are likely to be enhanced. For example, the more recent development of various immunotherapies is targeted to specifically elicit immune response against cells exposing neoepitopes that are mostly patient-specific and tumor-specific. While the individualized immunotherapy seems promising to treat many tumors that could not be effectively treated previously, effectiveness of such individualized immunotherapy often unexpectedly varies, and some patients may not at all develop a durable or protective immune response.

To address such difficulties, some have tried to predict immunogenicity by in vitro testing of immunogenic peptides. For example, WO2016/081,947 discloses identification of immunogenic neoepitopes by detecting intracellular cytokines in T cells reacting with the neoepitopes. In this example, specificity of the neoantigen reactive T cells is validated by detecting intracellular cytokines including IFNγ, and by detection of CD107a and MIP1β. However, such validation methodology is limited to characterize and detect limited types of immune cells, and cannot reliably and accurately reflect the status of immune response in the tumor as a whole. Moreover, such analysis also fails to account for immune suppressive cells in the tumor microenvironment.

Therefore, even though compositions and methods of individualized immunotherapy are known in the art, validation of such immunotherapy in a cost-effective, efficient, and relatively rapid manner is largely unexplored. Consequently, there is still a need for improved systems and methods that verifies and/or validates effectiveness of an intended individualized immunotherapy.

### SUMMARY OF THE INVENTION

The inventive subject matter is directed to various systems, compositions, methods and uses by which a patient's immune system in a tumor is examined using various cellular, molecular, and/or physiological parameters to assess the potential responsiveness of the immune system to one or more neoepitope-based cancer treatments (de novo or repeat treatment). In addition, based on the observed patient's immune system in the tumor, the individualized anticancer immunotherapy against a specific neoepitope can be adopted or excluded from the patient's treatment plan.

In one aspect of the inventive subject matter, systems and methods are contemplated to determine whether or not a patient's immune system is active or can be activated to mount a (preferably, therapeutically effective) immune response against tumor cells. Most preferably, the status of the immune system of a patient prior to neoepitope-based treatment may be determined by first obtaining a tissue sample (e.g., tumor, solid tumor, liquid biopsy sample (e.g., bodily fluid, blood), etc.) from the patient. From the tissue sample, at least one biochemical parameter can be quantitatively and/or qualitatively measured. For example, the biochemical parameter may include at least one pro-inflammatory chemokine, at least one pro-inflammatory cytokine, and/or at least one other pro-inflammatory marker (e.g., protein, RNA, DNA, post-translational modification, post-transcriptional modification, etc.). For example, such pro-inflammatory chemokine may include chemokine (C-X-C motif) ligand (e.g., CXCL1, CXCL4, CXCLS, CXCL6, etc.,), and pro-inflammatory cytokine may include one or more of cytokines such as interleukin molecules (e.g., IL-2, IL-12, IL-15, IL-18, IL-21, etc.) or tumor necrosis factor alpha (TNFα). In addition, the pro-inflammatory marker may include C-reactive protein (e.g., high-sensitivity C-reactive protein (hsCRP), etc.). Based on the measured biochemical parameter, a likely response status of the patient's immune system can be determined as a function of the measured quantities and the neoepitope-based treatment.

In addition, from the tissue sample, a prior response of the patient's immune system to the neoepitope can be measured, preferably by contacting the immune cells (e.g., immune competent cells, etc.) in the tissue sample to the neoepitope in vitro or ex vivo and determining the presence of the immune cells binding to the neoepitope. Then, the neoepitope-based treatment can be recommended/validated as a function of the prior response and the likely response status. Such method is particularly significant where a patient has already started chemotherapy and/or radiation therapy that may have reduced or even entirely abolished the capability of the patient's immune system to mount a durable or therapeutically effective immune response. Further, such method is useful to generate a detailed and reliable treatment plan where the patient already developed immune exhaustion against a specific neoepitope of the tumor or the progressive immune suppression in the tumor.

It is contemplated that the typical and preferred neoepitope-based treatment employs one or more neoepitopes having a length range from about 5 amino acids to about 35 amino acids (e.g., between 7-11 amino acids or between 12-25 amino acids, etc.). The neoepitope-based treatment may be administered as a vaccine, which may be a DNA or protein vaccine, or as a cell-based treatment that may include a transformed dendritic cell that expresses a neoepitope and optionally one or more of a chemokine, a cytokine, and/or checkpoint inhibitor. Also, it is contemplated that in some embodiments, a leukocyte profile can be analyzed as an indicator of active immune response or cell stress. Consequently, methods contemplated herein will also provide an indicated whether or not to administer a cell-based immune therapy (e.g., NK cell transfusion, CAR-T cell transfusion, etc.)

Viewed from a different perspective, the likely response status determines whether or not the patient's immune system will respond to an immune therapeutic treatment. To this end, the likely response status can be single-valued (e.g., based on a single cytokine), multi-valued (e.g., based on multiple cytokines and chemokines), a probability, a gradient, and/or a ratio. Most preferably, the likely response status is positive when the quantity of preferably at least two, more preferably at least three, and most preferably at least five of the chemokines and cytokines are above clinical reference range for normal cytokine or chemokine concentration. In another example, the likely response status could be positive when the quantity levels of one or more cytokines and/or chemokines are elevated in a tumor sample.

The effectiveness of the proposed neoepitope treatment is then validated as a function of the prior response and the likely response status. In some embodiments, and depending on the type of response status as determined above, the likely response status can be determined based on one or more of a threshold, a criterion, and/or a multi-factored response. Additionally, or alternatively, validation may also include a decision matrix plotting response status versus other factors relevant to immunotherapy, including the use of co-stimulatory factors, cell-based immunotherapy, and/or checkpoint inhibitors as part of the neoepitope-based treatment.

Another aspect of the inventive subject matter is directed towards determining whether the patient's immune-competent cells have already mounted a prior immune response to the neoepitope(s) in question (which may as such be indicative of exhaustion of a T cell response). To this end, the inventors contemplate a method of verifying an immune response to a tumor neoepitope in preparation of a treatment targeting the neoepitope in a patient. In this method, a tissue sample from a patient is obtained from a patient prior to the treatment of the patient. The tissue sample is exposed to the neoepitope, which is preferably is immobilized to a carrier, and then, at least one of antibodies, B cells, T cells, NK cells, and NKT cells that bind to the at least one immobilized neoepitope can be isolated. The presence of the isolated antibody, B cell, T cell, NK cells, or NKT cell binding to the neoepitope can be an indicative of the patient's immune system response to the neoepitopes, preferably the patient's prior exposure to the neoepitope and likely incomplete immune system response to the neoepitopes. As will be readily appreciated, depending on the type of test, the neoepitopes can be fixed to a bead, a plate, and/or a (e.g., colorimetric or fluorescent) substrate.

Still further, another aspect of the inventive subject matter is directed toward quantifying an immune response of a patient by analyzing a sample of the patient's blood under an immune stimulating condition. Exemplary methods includes (a) obtaining a blood sample from the patient; (b) exposing the blood sample to at least one neoepitope under immune-stimulating conditions, (c) quantifying the reaction of at least one of immune competent cells (B cells, T cells, NK cells, and/or NKT cells) in the blood sample (or other test system)to the at least one neoepitope; (d) characterizing the at least one of the immune competent cells with respect to a proportion in whole blood and/or with respect to immune competent cell subtypes, respectively, to generate a response metric. In this context, it should be noted that the blood sample may be whole blood, or a blood sample enriched in peripheral blood mononuclear cells (PBMCs) prior to exposing the cells to at least one neoepitope, and/or isolated PBMCs prior to exposing the cells to at least one neoepitope.

Typically, a greater proportion of neoepitope-reactive B cells indicate an activated immune response, and the presence of neoepitope-reactive T cells indicates a late-phase immune response. In addition, presence of neoepitope-reactive NK and/or NKT cells indicates a late-phase immune response against the neoepitope. The response metric is used to determine to exclude or include a neoepitope from the neoepitope-based treatment. For example, identification of neoepitope reactive NK cells is generally an indication to employ an immune therapy using that neoepitope and/or to employ a cell-based treatment that targets such neoepitope(s). In some embodiments, a plurality of patient's blood samples can be obtained by a predetermined time interval during a period of time, and each analysis on the blood sample can be used to generate a response metric. Thus, in these embodiments, the response metric may represent a change of immune response of the patient over a period of time. As before, such method is not only significant in predicting an anticipated immune response to an anticipated treatment, but may also be of importance to rule out immune therapy using a neoepitope against which the patient has already mounted an ineffective immune response.

There are numerous immune-stimulating conditions known in the art (e.g., exposing the blood sample to a lipopolysaccharide, a bacterial endotoxin, and/or one or more cytokines), and all of those are deemed suitable for use herein. Moreover, where contemplated methods include cultivation of cells or cell fractions, all manners of cell cultivation are also contemplated herein.

Various objects, features, aspects and advantages of the inventive subject matter will become more apparent from the following detailed description of preferred embodiments.

### DETAILED DESCRIPTION

Many tumor cells express a variety of neoepitopes that may arise from a mutation, overexpression of a subtype, specific splicing variants, of one or more (tumor-related) genes, and various tools and methods of individualized immunotherapy targeting such neoepitopes are known in the art. However, the inventors found that not all neoepitopes are effective to elicit immune responses, and especially therapeutically effective immune responses in the tumor microenvironment of the patient. In addition, the inventors further found that some neoepitopes that can effectively elicit immune response in some patients may not be effective in some other patients. Moreover, the inventors noted that indications of a prior ineffective immune response (e.g., due to anergic T cells, or checkpoint inhibition) may reveal that a patient's immune system already targeted a specific neoepitope.

Thus, the inventors now contemplate that a patient's immune system can be assessed using one or more molecular, cellular, biochemical, or physiological parameters that can indicate whether the patient's immune system, especially that of the tumor microenvironment is “hot” or “cold” to one or more neoepitope-based treatment. The immune system is “hot” if the immune system with respect to the tumor is active or activatable, especially to the neoepitope expressed on the tumor. Conversely, the immune system is “cold” if the immune system with respect to the tumor is inactive or suppressed.

For example, in one aspect of the inventive subject matter, the inventors contemplate that the assessment of the patient's immune system can be used for validation of an anticipated immune therapeutic treatment, and especially for validation of a neoepitope-based treatment. More specifically, the tumor (microenvironment) in the context of a “hot” immune system is likely to be susceptible to the one or more neoepitope-based treatment and that the tumor (microenvironment) in the context of a “cold” immune system is less likely to be susceptible to the one or more neoepitope-based treatment. Viewed from a different perspective, the inventors contemplate that effectiveness of the immunotherapy can be predicted by assessing various attributes of the immune system of the patient, and specifically responsiveness to the one or more neoepitope. Thus, the inventors contemplate that prior to the administration of a neoepitope-based cancer treatment, assessment of the patient's immune system status can provide guidance if a neoepitope-based therapy will likely have a desirable therapeutic effect, or in other words, whether the neoepitope-based therapy should be included (adopted) or excluded (rejected).

As used herein, the term “tumor” refers to, and is interchangeably used with one or more cancer cells, cancer tissues, malignant tumor cells, or malignant tumor tissue, that can be placed or found in one or more anatomical locations in a human body. As used herein, the term “bind” refers to, and can be interchangeably used with a term “recognize” and/or “detect”, an interaction between two molecules with a high affinity with a K_{D} of equal or less than 10⁻⁶ M, or equal or less than 10⁻⁷ M. As used herein, the term “provide” or “providing” refers to and includes any acts of manufacturing, generating, placing, enabling to use, or making ready to use.

### Profiling of Patient's Immune System

Assessing a patient's immune system can be done in a number of ways, including: (a) assessing whether the patient's immune system with respect to the tumor is active or activatable (“hot”) or inactive or suppressed (“cold”); (b) identifying a prior response of immune-competent cells against selected neoepitopes in the patient; and/or (c) determining a capability of immune-competent cells in the patient to respond to a particular neoepitope.

It is contemplated that the effectiveness of a neoepitope-based treatment may depend, at least in part, on whether a patient's immune system is responsive or active (e.g. “hot”) to a tumor. Moreover, prior immune response to a neoepitope may be indicative of a suppressed immune response (e.g., due to presence of Tregs, M2 macrophages, and/or myeloid derived suppressor cells (MDSC), one or more checkpoint inhibitors, and/or immune suppressing cytokines such as IL-8 or TNF-β, etc.). The activeness and/or responsiveness of the immune system can be determined by one or more parameters, including, but not limited to molecular parameters, biochemical parameters, cellular parameters, and physiological parameters that may change upon the activation or inactivation of the immune system. Thus, in one especially preferred aspect of the inventive subject matter, the inventors contemplate that a patient's tissue sample can be obtained and the status of the immune system in the patient's tissue sample can be determined using biochemical parameters.

Most typically, the patient's tissue sample is a tumor sample that is obtained from the patient through a biopsy of the tumor tissue during surgery or regular biopsy procedure. The tumor sample may include at least a portion of a solid tumor or non-solid tumor. In other embodiments, the patient's tissue sample includes bodily fluids or any sample derived from the bodily fluids (e.g., whole blood, plasma, serum, saliva, ascites fluid, spinal fluid, urine, etc.), which may include molecules and/or cells indicative of status of the immune system, and especially various immune mediators and/or cell free nucleic acids (e.g., cfRNA, cfDNA, preferably from tumor cells).

The patient's tissue can be used as fresh tissues (e.g., within 30 min, within 1 hour, within 2 hours, within 3 hours, within 6 hours after biopsy) or preserved tissues (e.g., quick-frozen, etc.). While it is preferred that the patient's tissue is used without any modulation or modification to reflect the immune system in vivo more accurately, it is also contemplated that the patient's tissue can be processed in a manner minimizing disruption of the immune system of the patient's tissue. For example, in one embodiment, the biopsy tumor tissue can be processed via manual and/or automated tissue slicing (e.g., using Leica Vibratome, etc.) under semi-sterile conditions, into acutely sliced tissues with a thickness of 100-500 μm, preferably 150-400 μm, and more preferably about 150-300 μm, and most preferably about 200-250 μm per each slice. The acutely sliced tumor tissue can be placed on a chamber for any biochemical analysis within 10 min, within 30 min, or within 1 hour after the slices are generated. Additionally, the acutely sliced tumor tissue can be moved to a chamber containing tissue culture media (e.g., Dulbecco's modified eagle media (DMEM)-based tissue culture media, Gibco® RPMI-1640-based media, etc.), for example, to be cultured in 37° C., 5% CO₂ environment for at least 6 hours, at least 12 hours, at least 2.4 hours, at least 3 days, at least 7 days, any biochemical analysis.

In some embodiment, the entire biopsy tumor tissue or the tumor tissue slice can be used. In other embodiments, the biopsy tumor tissue or the tumor tissue slice can be further processed into (e.g., punched, etc.) smaller tissues pieces (e.g., less than 2 mm×2 mm, less than 1.5 mm×1.5 mm, less than 3 mm diameter, less than 2 mm diameter, etc.). In such embodiments, the inventors contemplate that the locations of the smaller tumor tissues can be mapped relative to the entire or at least a portion of the biopsy tissue to determine or identify any heterogeneity in status of immune system (e.g., hot or cold status) among sub-regions of the tumor.

With respect to the parameters, the inventors contemplate any suitable parameters that may indicate the activity or status of a patient's immune system. In some embodiments, the parameters may include molecular parameters, biochemical parameters, cellular parameters, and physiological parameters. The molecular parameters may include any genomics and/or transcriptomics-derived parameters including, but not limited to, genetic mutations that may affect the expression of tumor-related genes or inflammation-related genes. The biochemical parameters may include presence, expression and/or activity level of any protein, non-protein molecules (e.g., secreted protein, cellular protein, extracellular matrix, lipid, glycolipid, nitrogen oxide, etc.) that may directly or indirectly regulate the patient's immune system or directly or indirectly be affected by the patient's immune system. The cellular parameters may include presence and/or activity level of any cells, preferably immune cells, in the patient's body, preferably in the tumor microenvironment. Thus, the cellular parameters may include the quantity, location, and/or interaction of leukocytes (e.g., neutrophil, eosinophil, basophil, lymphocyte, etc.) or more specifically, lymphocytes and/or monocytes, and more specifically, immune competent cells (e.g., T cells, B cells, NK cells, NKT cells, etc.), antigen presenting cells (e.g., dendritic cells, etc.), and immune suppression cells (e.g., myeloid-derived suppressor cells (MDSC), regulatory T cells (Treg cells), etc.). The physiological parameters may include any physiological signs that may be related to immune activity or inflammation, including, but not limited to, body temperature, blood pressure, oxygen levels, or any other vital signs.

In a preferred embodiment, the biochemical parameter includes quantitative (e.g., absolute amount, ratio, concentration, etc.) and/or qualitative (e.g., ratio, potency, etc.) profiles of tumor-associated or inflammation-related molecules including secretory proteins such as pro-inflammatory cytokines or chemokines, and other inflammation-induced protein/peptide markers. For example, For example, one approach could analyze a patient's liver tumor sample for the presence of cytokines such as interleukin 18 (IL-18), IL-1A, and leptin, as well as the presence of chemokines C-C motif chemokine ligands-2 (CCL-2), CCL-3, and CCL-5, while also searching for the presence of high sensitivity C-reactive protein (hsCRP) in a whole blood sample from the patient.

Of course, the inventive subject matter is not limited to cytokine IL-18, IL-1A, leptin, and chemokines CCL-2, CCL-3, and CCL-5, but will include various other cytokines, chemokines, and/or inflammatory markers, most preferably pro-inflammatory cytokines and chemokines, which may be secreted or cell-bound. Thus, pro-inflammatory cytokines including, but not limited to, IL-18, IL-1A, IL-1B, IL-1F10, IL-1F6, IL-1F8, IL-1RL2, IL-1F9, IL-33, cardiotrophin-like cytokine factor 1(CLCF1), IL-11, IL-31, IL-6, leptin, oncostatin M (OSM), B cell activation factor (BAFF), tumor necrosis factor (TNF) family proteins including TNF-SF8, TNFα, CD-40 ligand (e.g.,CD40LG), CD70, leukotriene family proteins (e.g. LTB, etc.), and galectin-3, are contemplated for use herein. Likewise, the use of all known pro-inflammatory chemokines, including, but not limited to, CCL-2, CCL-3, CCL-5, CXCLn (as n denoting number, e.g., CXCL-1, CXCL-2, CXCL-8, etc.) are also contemplated. Similarly additional markers of inflammation will include C-reactive protein (CRP), high-sensitivity C-reactive protein (hsCRP), fibrinogen, ferritin, and any other proteins or peptides that quantitatively or qualitatively change or alter upon, before, or even after onset of inflammation.

Any suitable methods or assay protocols to quantitatively and/or qualitatively measure any changes of such tumor-associated or inflammation-related molecules are contemplated. In at least some embodiments, a protein extract (e.g., whole cell extract, membrane extract, cytoplasmic extract, etc.) of the patient's tissue can be generated to measure the quantity and/or quality of inflammation-related molecules in or at the tissue. In other embodiments, the patient's tissue are placed on tissue culture plate (e.g., tissue culture insert, etc.) and incubated at least 1 hour, 3 hours, 6 hours, 12 hours, or 24 hours before the culture medium is collected to measure the quantity and/or quality of inflammation-related molecules secreted from the tissue to the culture medium. One or more cytokines, chemokines, and/or inflammatory markers' quantity can be assessed using enzyme-linked immunosorbent assay (e.g., multi-analyte ELISArray™, etc.), western blotting, and/or mass spectrometry. In some embodiments, such assays can be used to qualitatively detect various subtypes of inflammation-related molecules including various post-translational modification, protein-protein interaction, and so on.

The inventors contemplate that potential immune response may be determined by calculating profiles of the patient's pro-inflammatory cytokines, chemokines, and/or other markers from a patient sample. Calculating these profiles could allow the deduction of on-going conditions indicative of an immune response to a tumor in a patient, as well as the potential for possible activation. It is contemplated that the profile could indicate a general baseline of the patient. For example, when and if the immune system is “hot”, pro-inflammatory cytokines, chemokines, and/or other markers in the tumor environment and/or whole blood may be elevated relative to clinically ‘normal’ reference values or reference ranges (e.g., at least 10%, at least 20%, at least 30%, at least 40%, at least 50%, etc.), which could serve as an indicator that the tumor may be susceptible to the immune response against the tumor, or that the strength of the immune response against the tumor that can be elicited by immunotherapy can be strong. For other example, when and if the immune system is “cold”, pro-inflammatory cytokines, chemokines, and/or other markers in the tumor environment and/or whole blood may be substantially same (e.g., within 5% high or low, within 10% high or low, etc.) or even lower (e.g., at least 10%, at least 20%, at least 30%, at least 40%, at least 50%, etc.) relative to clinically ‘normal’ reference values or reference ranges, which may indicate that the tumor may be resistant to the immune response against the tumor, or that the strength of the immune response against the tumor that can be elicited by immunotherapy can be weak or insignificant.

Preferably, in such embodiments, the immune status as “hot” or “cold” can be determined by the quantity of two or more, preferably at least three, more preferably at least four, most preferably at last five of the cytokines or chemokines. For example, the immune status is determined “hot” when at least three or more cytokines or chemokines have quantity in the tumor sample at a level above the normal reference value. However, it is also contemplated that the immune status as “hot” or “cold” can be determined by the quantity of one or two most dominant cytokines or chemokines that may govern the immune status of the patient. Alternatively, the immune status as “hot” or “cold” can be determined the ratio of cytokines or chemokines with quantities at or above the normal reference value.

One should appreciate that the contemplated profiles may not be limited to absolute quantity or concentration of the inflammation-related molecules, but may comprise one or more dimensions of relevance, including temporal factors, location factors (e.g., position on a body, etc.), calculated values (e.g., ratios, amounts, concentrations, gradients, etc.), or other factors. For example, high amount of local TNF-a compared to the clinically ‘normal’ reference values (e.g., at least 50% higher, at least 70% higher, at least 100% higher, etc.) may be an indicator of acute immune response in the tumor microenvironment. In contrast, sustained low amount of local TNF-α compared to the clinically ‘normal’ reference values (e.g., within 10% of the normal range, etc.) can be a signature of tumorigenesis or even escaping from the immune response. For other example, where the quantity of one or more cytokines in one tumor sample (e.g., tumor biopsy sample) is higher than the quantity of one or more cytokines in the blood (e.g., liquid biopsy), it may indicate that the immune system is only locally activated in the tumor microenvironment and locally applied neoepitope-based treatment may be effective. Conversely, where the quantity of one or more cytokines in one tumor sample (e.g., tumor biopsy sample) is lower than the quantity of one or more cytokines in the blood (e.g., liquid biopsy), it may indicate that the immune system is only locally suppressed in the tumor microenvironment and locally applied neoepitope-based treatment may not be effective. Alternatively, systemically high cytokine level compared to local cytokine level at the tumor may also indicate other immune activating condition (e.g., systemic inflammation or medical condition other than tumor, etc.) that may render the individualized immunotherapy less effective when administered.

The inventors further contemplate that the effectiveness of a neoepitope-based cancer treatment may depend on the prior response mounted by a patient's immune-competent cells. If a patient's immune-competent cells have previously mounted an immune response to a neoepitope, a treatment regimen based on that neoepitope may likely fail, at least without one or more treatments to further stimulate the patient's immune response. Thus, a prior immune response (and especially therapeutically ineffective immune response that did not lead to reduction or inhibition of tumor growth or tumor shrinkage) to the neoepitope in the patient can be assessed to determine or validate whether any neoepitope-based treatment would be effective to treat the tumor in the patient and/or whether additional immune stimulation by administering immune stimulatory molecules would be necessary to aid the neoepitope-based treatment. For example, presence of anergic T cells with respect to a specific neoepitope may be indicative of immune exhaustion and may as such be indicative of likely treatment failure of an immune therapy targeting that specific neoepitope. Similarly, memory T or NK cells with respect to a specific neoepitope may be indicative of a prior immune response and may as such be indicative of likely treatment failure due to clonal depletion of cells producing that specific neoepitope. On the other hand, presence of T cells that are ex vivo responsive to the specific neoepitope may be indicative of a likely treatment success where immune suppressor cells or immune suppressive cytokines are also present in a tumor (microenvironment).

In one especially preferred aspect of the inventive subject matter, the inventors contemplate that a patient's tissue sample (e.g., tumor sample, blood sample, body fluid sample, etc.) can be obtained. Optionally, the immune-competent cells (e.g., T cells, B cells, NK cells, NKT cells, etc.) and/or cell products (e.g., antibodies, etc.) can be isolated from the patient sample for testing. Of course, it should be appreciated that the particular methods of extraction, enrichment, or other identification may vary depending on the type of tissue, immune-competent cell or antibody, and any suitable methods in the arts are contemplated. In some embodiments, the immune competent cells can be isolated from the tissue sample by the type (e.g., T cell, NK cell, NKT cell, B cell, etc.) or even by subtypes (e.g., CD4+ T cells, CD8+ T cells, CD1d-restricted NKT cells, etc.). Yet, it is also contemplated that the immune competent cells can be isolated from the tissue sample all together as leukocytes. Similarly, antibodies can be isolated from the tissue sample by the type (e.g., IgG, IgM, IgA, etc.).

Preferably, the patient's tumor sample may be examined to determine whether the neoepitope is expressed in a sufficient amount to lure the immune cells, and further whether neoepitope could properly expressed on the cell surface or not. Any suitable assays to determine the expression of neoepitope in the tumor tissue are contemplated, including, but not limited to, immunohistochemical assays, biochemical analysis assays (e.g., western blotting, etc.), and mass spectroscopy. Any presence of the neoepitopes, preferably sufficient level of surface expression of neoepitope on the tumor cell surface, more preferably at or above the detectable level with conventional biochemical or immunohistochemical assays, indicates that the patient's immune system has seen the neoepitope, is fighting it, or has ramped down a previous immune response.

The patient's tissue sample, or preferably the isolated immune-competent cells and/or cell products, are then subject to further analysis that includes, but not limited to, various solid phase analyses and/or liquid phase analyses with bound neoepitopes or labeled neoepitopes (e.g., fluorescence, luminescence, etc.), or in tissue samples with labeled neoepitopes or secondary antibodies. The solid phase analyses may include various binding assays including ELISA or Fluorescent in situ hybridization (FISH), and liquid phase analyses may include Fluorescence-activated cell sorting (FACS). Thus, in especially preferred embodiments, the cells or cell products binding to neoepitope are further isolated from the tissue sample, or the isolated immune-competent cells and/or cell products. Additionally, the isolated immune competent cells binding to the neoepitope can be further sorted to each cell type to determine the type, quantity, and/or specificity of the isolated immune-competent cells to the neoepitope.

For example, the isolated patient antibodies can be used as a proxy for B cells to determine if the patient has already had an immune response to the neoepitope(s) in question. In this context, it should be noted that the antibodies can be isolated or extracted from the patient sample, for example, by incubating the sample with bound neoepitopes in microwell plates or with magnetic beads carrying the neoepitopes. Finding an antibody responsive to a neoepitope can be indicative of a prior full-cascade response (e.g., involving dendritic cells, T cells, and/or B cells) in the patient against the neoepitope expressed on the tumor cell. Thus, if an antibody that binds to a neoepitope is found in the patient sample, it is likely that a prior immune response against the tumor cell via the neoepitope was incomplete, suppressed, or exhausted. Similarly, if B cells that are reactive to neoepitopes are found in the patient sample, it can be presumed that the patient's immune system had initiated at least a partial immune response to the neoepitopes. Similarly, reactive T cells, NK cells, and/or NKT cells to the neoepitope may indicate ongoing or past immune responses. Presence of Memory B cells and memory NK cells binding to the neoepitope indicate that the patient's immune system has seen and responded to the tumor cells expressing the neoepitope.

If the analysis focuses on analyzing the patient's immune-competent cells (as opposed to antibodies), the patient's immune-competent cells binding to the neoepitope can be characterized with respect to a proportion in whole blood, with respect to the cell type, or even with respect to the cell subtypes to assess the level, type, and/or phase of the patient's immune response. For example, if 0.001% or less of all T cells in the sample are identified as reactive T cells to the neoepitope, it is likely that there is no currently ongoing immune response against the tumor cell via the neoepitope. However, if, for example, 1% or more of all T cells, B cells, NK cells and/or NKT cells in the sample are identified as reactive T cells, B cells, NK cells and/or NKT cells to the neoepitope, it may indicate currently ongoing immune response against the tumor cell via the neoepitope and/or clonal expansion of T cells, B cells, NK cells and/or NKT cells upon the immune activation via the neoepitope. Further, based on the ratio or activity of the T cells, B cells, NK cells and/or NKT cells at a given time may indicate the phase and/or future direction of the immune response of the patient against the tumor (via neoepitope) such that the treatment regime can be validated based on the expected future direction.

More specifically, a synthetic neoepitope peptide can be used can be used to isolate B cells with membrane bound antibodies specific to the neoepitope, or secreted antibodies generated from B cells that are specific to the neoepitope. It is contemplated that quantity of B cells can be measured (a) with respect to a proportion in whole blood (including any and all detectable immune cells in the blood), (b) with respect to all B cell types, and/or (c) with respect to B cell subtype (e.g., plasma cell, memory B cell, regulatory B cells, etc.), to generate a response metric. The inventors contemplate that a greater proportion of B cells other than regulatory B cells in the whole blood (or in the tissue sample) indicates an activated immune response, and increased proportion of regulatory B cells indicates the suppressed immune response in the tumor microenvironment. Thus, the B cell response metric can provide a guidance whether a neoepitope-based treatment can be included or excluded from the treatment. For example, where the B cell response is active against the neoepitope, a DNA vaccine encoding the neoepitope peptide may be an effective tool to boost the immune response against the tumor expressing the neoepitope. However, where the B cell response is relatively inactive, a DNA vaccine encoding the neoepitope peptide may be excluded from the treatment option, and other treatment options including cell-based neoepitope-based treatment may be considered alternatively.

With respect to NK cells or NKT cells, a synthetic neoepitope peptide can be used can be used to isolate NK cells and/or NKT cells specific to the neoepitope. Similar to B cells, it is contemplated that quantity of NK cells or NKT cells can be measured (a) with respect to a proportion in whole blood (including any and all detectable immune cells in the blood), (b) with respect to all NK cell types or NKT cell types, respectively and/or (c) with respect to NK cell subtypes (e.g., CD56^{dim} CD16⁺, CD56^{bright}CD16^{+/-} and CD56⁻CD16⁺, etc.) or NKT cell subtypes (e.g., CD1d-restricted Type 1 NKT, CD1d-restricted Type 2 NKT, etc.) respectively, to generate a response metric. It is contemplated that the high proportion of NK cells and/or NKT cells specific to the neoepitope among the entire NK cells and/or NKT cells in the tissue sample may indicate the active ongoing immune response against the tumor cells expressing the neoepitope as an antigen. Also, typically, the presence of neoepitope-reactive NK cells or NKT cells indicates a late-phase immune response.

In addition, the inventors contemplate that the activity of NK cells and/or NKT cells against the cells expressing the neoepitope can be examined by exposing the NK cells and/or NKT cells to the synthetic neoepitope peptide ex vivo. For example, NK cells and/or NKT cells can be exposed to bound neoepitopes (e.g., bound to solid substrate) or antigen presenting cells (e.g., dendritic cells, etc.) expressing the neoepitopes to determine a response of the so treated NK cells or NKT cells. Observation of NK cell and/or NKT cell reactions to the neoepitopes or neoepitope-bound cells (e.g., via microscopy or via biochemical assays) could indicate the status and level of immune response against the neoepitope. For example, the patient's NK cells could be exposed to beads or microwells that have been decorated with neoepitopes then exposed to synthetic antibodies that bind to the neoepitopes. The activity and/or potency of patient's NK cells against the neoepitope may then be observed by detecting released amount of granzyme and/or perform or amount of cytokines (e.g., IFN-γ and TNF).

With respect to T cells, a synthetic neoepitope peptide can be used can be used to isolate NK cells and/or NKT cells specific to the neoepitope. Additionally, the entire or a t least a portion of entire population of T cells (e.g., specific types of T cells, etc.) can be isolated from patient blood using various markers (e.g., CD4, CD8, etc.). Alternatively, T cells can be isolated directly from blood using a T cell receptor. There are numerous methods of T cell isolation known in the art, and all are deemed suitable for use herein. It is contemplated that quantity of T cells can be measured (a) with respect to a proportion in whole blood (including any and all detectable immune cells in the blood), (b) with respect to all T cell types, respectively and/or (c) with respect to T cell subtypes (e.g., CD4+ T CD8+ T cell, regulatory T cells, etc.) respectively, to generate a response metric. The inventors contemplate that a greater proportion of T cells other than regulatory T cells indicates an activated immune response, and increased proportion of regulatory T cells indicates the suppressed immune response in the tumor microenvironment. Also, typically, the presence of neoepitope-reactive T cells indicates a late-phase immune response.

Additionally, the inventors contemplate that the activity of T cells against the cells expressing the neoepitope can be examined by exposing the T cells to the synthetic neoepitope peptide ex vivo. After isolation, isolated T cells can be exposed to bound neoepitopes (e.g., bound to solid substrate) or antigen presenting cells (e.g., dendritic cells, etc.) expressing the neoepitopes to determine the immune response (e.g., antibody-dependent cell-mediated cytotoxicity (ADCC), etc.). ADCC could be determined using live cells, or using beads or other solid structures to which neoepitopes are bound. Such beads or other solid structure may further include substrates that are reactive to the enzymes released during ADCC, for example, chromogenic perform or granzyme substrates. Upon release of the lytic enzymes, ADCC can be directly observed in qualitative or even quantitative manner (e.g., using microspectrophotometry. Thus, observation of T cell reactions to the neoepitopes or neoepitope-presenting cells (e.g. via microscopy or otherwise) could indicate an immune response.

The inventors contemplate that T cell response metric can provide a guidance whether a neoepitope-based treatment can be included or excluded from the treatment. For example, where the T cell response is active against the neoepitope, a DNA vaccine encoding the neoepitope peptide may be an effective tool to boost the immune response against the tumor expressing the neoepitope. Further, NK or NKT cell-based neoepitope-based treatment may be considered additionally with the DNA vaccine or as an alternative treatment option. However, if the proportion of the regulatory T cells is relatively high, then a DNA vaccine encoding the neoepitope peptide may be excluded from the treatment option, and other treatment options including cell-based neoepitope-based treatment with immune stimulatory molecule to remove immune suppression may be considered alternatively.

The inventors further contemplate presence of different types of T cells may validate different types of treatment options. For example, if memory T cells specific to the neoepitope are present in the tumor sample, it may indicate that the immune system has previously responded to the neoepitope. In this case, a checkpoint inhibitor therapy can be a recommended treatment option for the patient. For other example, if regulatory T cells are dominantly present or at least the proportion of the regulatory T cells is abnormally high compared to the normal tissue (of healthy individual), the neoepitope-based therapy is expected to be ineffective or less effective, thus can be excluded from the treatment options. Instead, it is contemplated that a treatment option with immune stimulatory cytokines may be recommended to reactivate exhausted immune cells. Alternatively, exhausted (i.e. previously active) T cells may be isolated, propagated, and reactivated for reintroduction into the patient.

Further, the inventors contemplate that some immune suppression-related cells can be profiled in the tumor tissue. For example, human MDSCs can be isolated from the tumor tissue or from the blood using a commonly expressed marker Siglec-3/CD33. In some embodiments, heterogeneous group of MDSCs can be further sub-divided and isolated using CD14 or CD15. It is contemplated that quantity of MDSCs can be measured (a) with respect to a proportion in whole blood (including any and all detectable immune cells in the blood), (b) with respect to all MDSC cell types, or (c) with respect to MDSC cell subtype (e.g., CD14+ or CD15+ cells, etc.), and/or (d) with respect to all detectable immune cells in the tumor tissue (non-blood tumor tissue). The inventors contemplate that a greater proportion of MDSCs in the tumor tissue or blood indicates increased and/or substantial immune suppression in the tumor microenvironment. Thus, the MDSC (or other immune suppression-related cells) response metric generated based on the MDSC profile can provide a guidance whether a neoepitope-based treatment can be included or excluded from the treatment. For example, where MDSC is active and immune response is suppressed in the tumor microenvironment, a DNA vaccine or cell-based treatment may not be effective without co-treatment of immune stimulators including one or more cytokines.

### Neoepitope-Based Treatment

With respect to the neoepitope-based treatment, candidate neoepitopes can be identified from the omics data obtained from the cancer tissue of the patient or normal tissue (of the patient or a healthy individual), respectively (for example, by using the process described in U.S. Patent Application 2012/0066001). Omics data typically includes information related to genomics, transcriptomics, and proteomics. While it is contemplated that any and all neoepitopes are deemed suitable for neoepitope-based treatment, it is especially preferred that the neoepitope is derived from the tumor-associated gene, and even cancer-driving gene. Thus, in some embodiments, the neoepitopes can also be cancer specific antigens, for example PSA or HER2, or any tumor associated antigen (e.g., CEA1, MUC1, etc.). Preferably, the neoepitopes are also MHC-matched to the specific patient so that they are presented to the patient's immune system via MHC the patient's MHC protein complexes. Thus, typically and preferably, the neoepitope used in the neoepitope-based treatment is patient-specific and tumor-specific. Further it is highly preferred that neoepitopes chosen for the neoepitope-based treatment are those transcribed at or above normal transcription and/or translation in the tumor tissue such that the neoepitopes can be well presented on the tumor cell surface, which then be targeted by one or more neoepitope-based treatment.

It is contemplated that neoepitope-based treatment can include cancer vaccine comprising the neoepitope. For example, cancer vaccine may include a genetically engineered microorganism (e.g., recombinant virus, bacteria or yeast) having recombinant nucleic acid encoding one or more neoepitopes (e.g., as a polytope, etc.). Any suitable expression vectors that can be used to express protein are contemplated. Especially preferred expression vectors may include those that can carry a cassette size of at least 1 k, preferably 2 k, more preferably 5 k base pairs. For example, where the microorganism is a virus preferred expression vector includes a viral vector (e.g., nonreplicating recombinant adenovirus genome, optionally with a deleted or non-functional E1 and/or E2b gene). For other example, where the microorganism is a bacteria, and the expression vector can be a bacterial vector that can be expressed in a genetically-engineered bacterium, which expresses endotoxins at a level low enough not to cause an endotoxic response in human cells and/or insufficient to induce a CD-14 mediated sepsis when introduced to the human body. One exemplary bacteria strain with modified lipopolysaccharides includes ClearColi® BL21(DE3) electrocompetent cells. Alternatively, or additionally, the microorganism is a yeast, and the expression vector can also be a yeast vector that can be expressed in yeast, preferably, in Saccharomyces cerevisiae (e.g., GI-400 series recombinant immunotherapeutic yeast strains, etc.).

In other embodiments, the neoepitope-based treatment may include cell-based treatments such as genetically-modified antigen presenting cells or other cytotoxic or cytolytic immune competent cells. For example, in one embodiment, antigen presenting cells (e.g., dendritic cells) can be modified by recombinant peptide including neoepitope such that the antigen presenting cells can present the neoepitope on their surface. The modified antigen presenting cells can be directly administered to the patient to elicit patient's immune response against the neoepitope. Alternatively, the modified antigen presenting cells can be used to activate immune-competent cells including T cell, NK cell, NKT cells (derived preferably from the patient's blood), and then the activated immune-competent cells can be administered to the patient to boost patient's immune response against the neoepitope.

For other example, at least one or more T cells, NK cells, or NKT cells can be genetically engineered to express recombinant neoepitope-specific receptors. Generally, the recombinant protein is a CAR and includes an extracellular single-chain variant fragment, an intracellular activation domain, and a transmembrane linker coupling the extracellular single-chain variant fragment to the intracellular activation domain. Preferably, the recombinant protein is generated from a single chimeric polypeptide translated from a single recombinant nucleic acid. However, it is also contemplated that that the recombinant protein comprises at least two domains that are separately translated from two distinct recombinant nucleic acid such that at least a portion of the recombinant protein can be reversibly coupled with the rest of the recombination protein via a protein-protein interaction motif.

Alternatively, the inventors also contemplate that the extracellular single-chain variant fragment (V_{H} and/or V_{L} chains) can be substituted with an extracellular domain of T-cell receptor. For example, in some embodiments, the extracellular single-chain variant fragment can be substituted with a portion of α chain, β chain, γ chain, or δ chain of T cell receptor. In other embodiments, the extracellular single-chain variant fragment can be substituted with a combination of at least two of a portion of α chain, β chain, γ chain, or δ chain (e.g., hybrid of α chain and β chain, a hybrid of γ chain and δ chain, etc.) of T cell receptor. In this embodiment, the nucleic acid sequence of extracellular domain(s) of T-cell receptor, especially hypervariable region(s) of α, β, γ and/or δ chain can be selected based on the measured, estimated, or expected affinity to the tumor neoepitope, tumor associated antigen, or self-lipid. In still other embodiment, the extracellular single-chain variant fragment can be substituted with extracellular domain of the CD1d-restricted NKT cell receptor (e.g., Vα24-Jα18). It is especially preferred that the affinity of extracellular domain of T-cell receptor or CAR to the neoepitope, is at least with a K_{D} of at least equal or less than 10⁻⁶ M, preferably at least equal or less than 10⁻⁷ M, more preferably at least equal or less than 10⁻⁸ M.

In addition, neoepitope-based treatments may be assisted by other immune activating compounds including, but not limited to, co-stimulatory molecule, an immune stimulatory cytokine, and/or a protein that interferes with or down-regulates checkpoint inhibition. Suitable co-stimulatory molecules include CD80, CD86, CD30, CD40, CD30L, CD40L, ICOS-L, B7-H3, B7-H4, CD70, OX40L, 4-1BBL, while other stimulatory molecules with less defined (or understood) mechanism of action include GITR-L, TIM-3, TIM-4, CD48, CD58, TL1A, ICAM-1, LFA3, and members of the SLAM family However, especially preferred molecules for coordinated expression with the cancer-associated sequences include CD80 (B7-1), CD86 (B7-2), CD54 (ICAM-1) and CD11 (LFA-1). In addition, suitable checkpoint inhibitors will inhibit or at least reduce signaling via the receptor, and particularly contemplated receptors include CTLA-4 (especially for CD8⁺ cells), PD-1 (especially for CD4⁺ cells), TIM1 receptor, 2B4, and CD160. For example, suitable peptide binders can include antibody fragments and especially scFv, but also small molecule peptide ligands (e.g., isolated via RNA display or phage panning) that specifically bind to the receptors. Furthermore, the immune stimulatory cytokine may include IL-2, IL-12, IL-15, IL-15 super agonist (ALT803), IL-21, IPS1, and LMP1.

### Validation of Treatment Options

In general, the inventors contemplate that a patient with elevated levels of pro-inflammatory cytokines, chemokines, and/or other pro-inflammatory markers could be considered a good candidate for a neoepitope-based treatment because the immune system is already “hot” or “ramped up,” (i.e. ready to respond, etc.). In contrast, a patient with high levels of immune suppressor molecules or molecules related to checkpoint inhibition, concentrated presence of immune suppressor cells (e.g., Treg cells, MDSCs, etc.) in the tumor microenvironment could indicate the patient's immune system is “cold,” or not ready to mount an immune response because a response has already been made and then deactivated. Thus, in some embodiments, a “cold” status of the patient's immune system may indicate that the treatment regimen should include additional immune stimulating cytokines, checkpoint inhibitors, and/or agents that reduce Tregs and MDSCs.

The inventors further contemplate that the likely immune response can be more reliably inferred or determined when the profile of the patient's immune system (e.g., quantity of pro-inflammatory cytokine, etc.), the types of neoepitope-based treatment, and prior immune response to one or more neoepitopes intended for treatment are considered in combination. For example, there is a likely immune response (i.e. a positive likely response status) where the neoepitope-based treatment is cell-based and the expression level or secreted amounts of pro-inflammatory markers (e.g., pro-inflammatory cytokines, inflammation-related peptide, etc.) are high (e.g., at least 10% higher, at least 20% higher, at least 30% higher, at least 40% higher, etc. compared to the reference value), and no prior immune response to the neoepitopes has been observed in the patient. In another example, where the neoepitope-based treatment is a vaccine, normal (e.g., within 5%, within 10% of the reference value) or elevated levels (5% elevated, 10% elevated, 15% elevated, 20% elevated, etc.) of expression or secreted amounts of pro-inflammatory markers, and/or no prior immune response to the neoepitope(s) may be indicative of a likely immune response. In yet another example, where the neoepitope-based treatment is antibody based, normal (e.g., within 5%, within 10% of the reference value) and low-levels of pro-inflammatory markers (e.g., 5% decreased or lower, 10% decreased or lower, 15% decreased or lower, 20% decreased or lower, etc. compared to the reference value) might be indicative of a likely therapeutic effect of the antibody based neoepitope-based treatment.

On the other hand, there is likely no therapeutically effective immune response (i.e. a positive likely response status) where the neoepitope-based treatment is cell-based and the expression level or secreted amounts of pro-inflammatory markers (e.g., pro-inflammatory cytokines, inflammation-related peptide, etc.) are normal or low (e.g., at or below 10% of reference value), and a prior immune response to the neoepitopes has been observed in the patient. In another example, when the neoepitope-based treatment is a vaccine, normal (e.g., within 5%, within 10% of the reference value) or elevated levels (5% elevated, 10% elevated, 15% elevated, 20% elevated, etc.) of expression or secreted amounts of anti-inflammatory markers, and/or a prior immune response to the neoepitope(s) may be indicative of a likely treatment failure using immune therapy. In yet another example, where the neoepitope-based treatment is antibody based, normal (e.g., within 5%, within 10% of the reference value) or low-levels of pro-inflammatory markers (e.g., 5% decreased or lower, 10% decreased or lower, 15% decreased or lower, 20% decreased or lower, etc. compared to the reference value), and a prior immune response to the neoepitope(s) might be indicative of a likely therapeutic effect of the antibody based neoepitope-based treatment.

Thus, the inventors contemplate that based on the likely immune response to a neoepitope-based treatment in the tumor and prior immune response in the patient, treatment options can be validated (i.e., a determination can be made whether such treatment options should be included or excluded from the treatment plan of the patient). In one preferred embodiment, the response status can be used to calculate a validity function of the neoepitope-based treatment. The validity function can be one or more of a threshold, a criterion, and/or a multi-factored response. For example, based on the patient's profile of the immune system that includes secreted amount (e.g., absolute amount per gram of tumor tissue, concentration, etc.) of two pro-inflammatory cytokines and two chemokines, the validity function can be calculated to generate one or more threshold values for treatment options. Additionally, the prior immune response of the patient can add or subtract some values from the threshold values such that the threshold values may be dynamic based on those two factors (patient's profile of the immune system and prior immune response). In such embodiment, at least one or more treatment options can be tagged with a value or a score, preferably predetermined value or score, which can be compared with the one or more threshold. If the value of one treatment option is lower than the threshold, it can be determined that that the treatment option is unlikely to be effective to treat the tumor in the patient and the treatment option may be excluded from the treatment plan. However, if the value of one treatment option is higher than the threshold, it can be determined that that the treatment option is likely to be effective to treat the tumor in the patient and the treatment option may be included in the treatment plan.

In another example, the validity function could comprise a decision matrix plotting the patient's immune status and the neoepitope-based treatment. In some embodiments, the patient's immune status can be a single-valued validity function that is calculated based on the patient's profile of the immune system. In another embodiment, each factor of the patient's profile of the immune system (e.g., quantity of each pro-inflammatory cytokines or chemokines, etc.) can be plotted separately on the same matrix. Further, the neoepitope-based treatment can be plotted as multiple points (or alternatively in n-dimensioned matrix, n=number of co-stimulatory factors and/or checkpoint inhibitors or combinations of co-stimulatory factors and/or checkpoint inhibitors) based on use of co-stimulatory factors and/or checkpoint inhibitors. In such embodiment, the treatment regimen may be determined or validated by the locations and/or distributions (e.g., vectors) of the treatment option(s) relative to the patient's immune status. For example, for each treatment option, the optimal conditions that the treatment option can be most effective can be determined, and the treatment option(s) can be plotted based on the range of optimal conditions (e.g., treatment option may be most effective when the pro-inflammatory molecule X quantity is ranged between A and B, and the treatment option is plotted in the axis of X between the value A and B, etc.). It is contemplated that the treatment option can be validated if the patient's immune status is overlapped at least 40%, at least 50%, at least 60%, at least 70%, or at least 80%, with the value range (e.g., between A and B) of the treatment option.

Additionally, the patient's profile of the immune system can be measured over time (e.g., every 1 hour, every 6 hours, every 12 hours, every 24 hours, every 3 days, every 7 days, etc.) to provide temporal values related to the measured quantiles. Based on such multiple measurements during a span of time, the likely responses of each time point can be inferred or determined. Preferably, each of the likely response can be coupled with a numerical value (either absolute or relative) such that the any changes of likely responses over time can be represented as one or more temporal values. Thus, the temporal values can be considered a time-based signature of the immune response. The temporal values (e.g., a trend line, a graph, a rate of change, etc.) can be compared to normal temporal values (e.g., of a healthy individual, etc.) to determine if a positive response is present. In addition, based on such multiple measurements during a span of time, prior immune responses over time can be inferred or determined, and a response metric can be generated based on the prior immune responses. Thus, the response metric can be considered as a representation of a change of the immune response of the patient.

It is further contemplated that in addition to a patient's immune status and the presence of a prior immune response by a patient's immune-competent cells, the capability for those immune competent cells to respond to a neoepitope-based treatment may be crucial to the effectiveness of that treatment. Thus, in one especially preferred embodiment, patient's immune-competent cells are analyzed to determine their ability to respond to the neoepitope or neoepitope-expressing cells. It should be noted that each of these analyses can be done with whole blood, or in a sample enriched for PBMC cells, or in a sample in which PBMC cells have been isolated (for example via Ficoll-Paque gradient centrifugation).

Generally, patient's blood sample or isolated immune competent cells (or mixture of several isolated immune competent cells) can be contacted ex vivo with at least one neoepitope under immune stimulatory conditions. Preferably, the at least one neoepitope may be immobilized on a solid substrate (e.g., a microwell plate, a bead, etc.) or expressed on a cell (e.g., antigen presenting cells such as dendritic cells, etc.). It is preferred that where the neoepitope is expressed on a cell, the cell is obtained and/or derived from the patient to match the MHC type. The duration for exposing the immune competent cells to the neoepitope may vary depending on the type of the immune competent cells and/or the physical/biochemical parameters to be detected upon the exposure. Thus, the immune competent cells can be exposed to the neoepitope for at least 5 min, at least 30 min, at least 1 hour, at least 3 hours, at least 6 hours, at least 12 hours, at least 24 hours, at least 3 days.

It should be appreciated that any physical, cellular or biochemical reactions or changes observable, detectable and/or quantifiable in an ex vivo environment can be used as an indication of likely response. For example, the reaction of various immune competent cells upon exposure to the neoepitope can be analyzed by detecting any physical, cellular changes (e.g., shape, volume, mass, density, etc.) of the immune competent cells. In some embodiments, such physiological changes can be directly observed under one or more different types of microscopy (e.g., via FISH, using live cell interferometry, real-time confocal microscopy, or time-stretch quantitative phase imaging, etc.). In another example, the reaction of various immune competent cells upon exposure to the neoepitope can be analyzed by detecting any biochemical reactions or changes including, but not limited to, detecting ADCC via quantifying the released amount of perform, granzyme, granulysin, or other cytokines (e.g., IL-2, TNF-α, etc.), or detecting activation of immune competent cells via detecting release of one or more cytokines from the cells to the nearby environment. On the other hand, B cells, T cells, NK cells, and/or NKT cells may be used as indirect markers of a likely immune response via the proliferation of reactive B cells, T cells, NK cells, and/or NKT cells. For example, a selective or overall expansion (e.g., clonal expansion) of these cells could be a strong indication that some cells were specifically reactive to the neoepitopes.

Additionally, the inventors contemplate that the types of T cells among memory T cells or cytotoxic T cells that are activated upon exposure to the neoepitope can be further identified and/or characterized. In some embodiments, once specific types of T cells that become active upon exposure to the neoepitope are identified, those active T cells can be further isolated and expanded ex vivo. Then, the expanded active T cells can be administered to the patient to boost the immune response against the tumor in the patient. In other embodiments, the identified, active T cell can be further analyzed to obtain the sequence of T cell receptor, especially the variable region of the T cell receptor, such that a chimeric T cell receptor, or a chimeric antigen receptor (CAR) can be generated using such sequence that are deemed or expected to be specific to the neoepitope and sufficient to activate the downstream signaling for T cell activation. Further, patient's immune cells (e.g., NK cells, NKT cells, etc.) can be genetically engineered to express such a chimeric T cell receptor or a chimeric antigen receptor (CAR) and further administered to the patient, especially to the tumor, to boost the immune response against the tumor in the tumor microenvironment.

Additionally, the ex vivo expanded immune competent cells and/or genetically engineered immune competent cells with a chimeric T cell receptor or a chimeric antigen receptor (CAR) can be further activated ex vivo before being administered to the patient. Any suitable method of generally or specifically activate immune competent cells are contemplated. For example, T cells can be activated ex vivo with co-incubating T cells with α-CD3 antibody or some cytokines including IL-2. In another example, NK cells can be activated ex vivo with co-incubating NK cells with α-PD-1 antibody, α-PD-L1 antibody, α-CTLA-1 antibody or some cytokines including IL-2. In still another example, NKT cells that are activated ex vivo with specific glycolipid agonist(s) (e.g., α-GlcCer, β-ManCer, GD3, etc.) depending on the desired immune response against the tumor cells or tumor microenvironment. Specifically, NKT cells activated with β-ManCer can be genetically modified as described above to induce NKT-cell mediated immune response that is dependent on NOS (e.g., involving macrophage activity, etc.). In yet another example, NKT cells activated with α-GlcCer can be genetically modified as described above to induce NKT-cell mediated immune response that is independent of NOS (e.g., suppression of MDSC-mediated immune suppression, etc.).

It should be appreciated that contemplated methods for assessing the patient's immune system and validating the neoepitope-based cancer treatments as treatment options can further be used to rank a plurality of neoepitopes and a plurality of neoepitope-based treatments available for each neoepitope. It is further contemplate that the rank of the a plurality of neoepitopes and a plurality of neoepitope-based treatments can be dynamically changed or updated where the patient's immune system is accessed a plurality of times and the status of the patient's immune system is changed or unchanged such that the treatment plan can be optimized based on the patient's response to the treatment and/or prognosis of the tumor.

### EXAMPLE

Patient Y is diagnosed with third stage non-small cell lung cancer, without any prior and current history of other immunodeficiency-related diseases. To design and prepare an individualized immunotherapy as a part of treatment options for Patient Y, tumor biopsy is performed and a small piece of tumor tissue is obtained from the biopsy. Through various omics analyses obtained from a portion of the obtained tumor tissue, neoepitopes A and B are found as tumor-specific and Patient Y-specific neoepitopes, and are also found as valid targets for individualized immunotherapy.

The rest of the tumor biopsy tissue, or additionally obtained biopsy tissue, preferably from the same tumor, is processed via an automated tissue slicing into acutely sliced tissues with a thickness of 200 μm per each slice. Some of the tissue slices are placed in a tissue culture chamber containing tissue culture media and incubated for 12 hours and supernatant (culture media) is collected. From the collected supernatant, the quantities of IL-18, IL-1A, CCL-2, CCL-3, and hsCRP are analyzed and compared with the normal reference value. The analysis results show that the concentration of IL-18, IL-1A, CCL-2 and hsCRP are at least 50-120% higher than the normal reference value. In addition, when the tissue slices are observed under the microscope with labeling the marker proteins for MDSC, the number of MDSC cells per 1 mm³ area is within 10% of normal MDSC distributions in non-tumor tissue. Based on such results, it can be determined that the likely response status of the Patient Y to cell-based immunotherapy is positive as the immune status of the patient Y in the tumor is “hot”.

Yet, neoepitope-based treatment using neoepitope A, B, or both may not be equally effective. In order to determine the likely efficacy of the neoepitope-based treatment, Patient Y's blood is obtained and PBMCs are enriched to generate the blood cell sample. The blood cell sample is divided into two groups to contact either neoepitope A or neoepitope B that are immobilized on a solid substrate. The immune cells binding to the neoepitope A or neoepitope B are isolated and further analyzed. Results show that a significant amount T cells and NK cells are bound to neoepitope A. However, no significant amount of immune cell is detected as being bound to neoepitope B. Such results will indicate that there is an ongoing immune response against neoepitope A but not neoepitope B in the patient's tumor, and that targeting neoepitope A may not be effective due to clonal depletion of cells producing that specific neoepitope.

Optionally, Patient Y's immune cells are tested to determine whether Patient Y's immune cells can be actively elicit immune response against the neoepitope B. For such analysis, Patient Y's blood is obtained and T cells, NK cells and/or NKT cells are isolated using the cell surface marker(s) of each cell types. The isolated cells are contacted with the immobilized neoepitope B or neoepitope B-expressing dendritic cells for at least 12 hours. The morphological changes of the cells are analyzed using live cell interferometry and/or confocal microscopy. The chemical changes of the cells are analyzed by western blotting of the cell extract and/or of cell medium (for secreted proteins, etc.).

Based on above, the treatment plan for Patient Y can be provided to include T cell or NK cell-based treatment where the T cell or NK cells are genetically engineered to target neoepitope B. It should be appreciated that the treatment plan for Patient Y could be different if prior immune responses were present against both neoepitope A and B. In such case, the treatment plan may include T cell or NK cells genetically engineered to target neoepitope A and/or B (preferably A and B as a polytope) with co-administration of co-stimulatory molecule, an immune stimulatory cytokine, and/or a protein that interferes with or down-regulates checkpoint inhibition.

It should be apparent to those skilled in the art that many more modifications besides those already described are possible without departing from the inventive concepts herein.

The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. Where the specification claims refers to at least one of something selected from the group consisting of A, B, C . . . and N, the text should be interpreted as requiring only one element from the group, not A plus N, or B plus N, etc.

What is claimed is:

1. A method of validating an anticipated neoepitope-based treatment of a patient diagnosed with cancer, comprising:
obtaining a tissue sample from the patient;
measuring quantities of at least one pro-inflammatory chemokine, at least one pro-inflammatory cytokine, and optionally at least one pro-inflammatory marker;
determining a likely response status of the patient's immune system as a function of the measured quantities and the neoepitope-based treatment;
measuring a prior response of the patient's immune system to the neoepitope; and
validating the neoepitope-based treatment as a function of the prior response and the likely response status.

2. The method of claim 1, wherein the neoepitope-based treatment includes treatment with one or more neoepitopes.

3. The method of claim 2, wherein the neoepitopes are patient- and tumor specific.

4. The method of any one of preceding claims, wherein the neoepitope-based treatment includes treatment with a vaccine.

5. The method of any one of preceding claims, wherein the neoepitope-based treatment includes treatment with a DNA vaccine.

6. The method of any one of preceding claims, wherein the neoepitope-based treatment includes treatment with a transformed dendritic cell which expresses a neoepitope and one or more of a chemokine, a cytokine, and/or checkpoint inhibitor.

7. The method of any one of preceding claims, wherein the tissue sample is one or more of a blood sample, a tumor sample, a solid sample, and/or a liquid biopsy sample.

8. The method of any one of preceding claims, wherein the pro-inflammatory chemokine is CXCLn.

9. The method of any one of preceding claims, wherein the pro-inflammatory cytokine is one or more of IL-1, IL-2, IL-12, IL-15, IL-18, IFN-γ, and/or IL-21.

10. The method of any one of preceding claims, wherein the pro-inflammatory marker is hsCRP.

11. The method of any one of preceding claims, wherein the likely response status is single-valued, multi-valued, a probability, and/or a ratio.

12. The method of any one of preceding claims, further comprising a step of analyzing a leukocyte profile.

13. The method of any one of preceding claims, wherein the likely response status is positive when the quantity of preferably at least two, more preferably at least three, and most preferable at least five of the chemokines and cytokines are above clinical reference range for normal.

14. The method of any one of preceding claims, wherein the prior response comprises detection of immune competent cells binding the neoepitope.

15. The method of claim 14, further comprising characterizing the at least one of the immune competent cells (a) with respect to a proportion in whole blood and/or (b) with respect to immune competent cell subtypes, respectively, to determine the prior response.

16. The method of any of claims 14-15, wherein the immune competent cells are at least one of B cells, T cells, NK cells, and NKT cells.

17. The method of any one of preceding claims, wherein the prior response comprises detection of an antibody binding the neoepitope.

18. The method of any one of preceding claims, wherein the likely response status is positive when the cytokine and/or chemokine are elevated in a tumor sample.

19. The method of any one of preceding claims, wherein the validity function is one or more of a threshold, a criterion, and/or a multi-factored response.

20. The method of any one of preceding claims, wherein the validity function comprises a decision matrix plotting status versus use of co-stimulatory factors and checkpoint inhibitors as part of the neoepitope-based treatment.

21. A method of verifying an immune response to a tumor neoepitope in preparation of a treatment targeting the neoepitope in a patient, comprising:
obtaining a tissue sample from a patient prior to the treatment of the patient;
exposing the tissue sample to the neoepitope, wherein the neoepitope is immobilized to a carrier; and
isolating at least one of antibodies, B cells, T cells, NK cells, and NKT cells that bind to the at least one immobilized neoepitope, wherein the presence of the isolated antibody, B cell, T cell, NK cells, or NKT cell is indicative of the patient's immune system response to the neoepitopes.

22. The method of claim 21, wherein the carrier is a bead, a plate, and/or a substrate, and wherein the tissue is whole blood, a white blood cell fraction, or tumor tissue.

23. A method of quantifying an immune response of a patient, comprising:
obtaining a blood sample from the patient;
exposing the blood sample to at least one neoepitope under immune-stimulating conditions;
quantifying the reaction of at least one of immune competent cells in the blood sample to the at least one neoepitope;
characterizing the at least one of the immune competent cells (a) with respect to a proportion in whole blood and/or (b) with respect to immune competent cell subtypes, respectively, to generate a response metric;
wherein the immune competent cells are at least one of B cells, T cells, NK cells, and NKT cells;
wherein a greater proportion of neoepitope-reactive B cells indicates an activated immune response, wherein the presence of neoepitope-reactive T cells, NK cells or NKT cells indicates a late-phase immune response; and
determining to exclude or include a neoepitope-based treatment from treatment options based on the response metric.

24. The method of claim 23, wherein the immune competent cells are B cells, and characterizing at least one of the immune competent cells comprises characterizing isolated B cells (a) with respect to a proportion in whole blood and/or (b) with respect to B cell subtype, to generate a response metric, wherein a greater proportion of neoepitope-reactive B cells indicates an activated immune response.

25. The method of any one of claims 23-24, wherein the immune competent cells are T cells, and characterizing at least one of the immune competent cells comprises characterizing isolated T cells (a) with respect to a proportion in whole blood and/or (b) with respect to T cell subtype, to generate a response metric, wherein the presence of neoepitope-reactive T cells indicates a late-phase immune response.

26. The method of any one of claims 23-25, wherein the immune competent cells are NK cells, and characterizing at least one of the immune competent cells comprises characterizing isolated NK cells (a) with respect to a proportion in whole blood and/or (b) with respect to NK cell type, to generate a response metric, wherein the presence of neoepitope-reactive NK cells indicates a late-phase immune response.

27. The method of any one of claims 23-26, wherein the immune competent cells are NKT cells, and characterizing at least one of the immune competent cells comprises characterizing isolated NKT cells (a) with respect to a proportion in whole blood and/or (b) with respect to NKT cell subtype, to generate a response metric, wherein the presence of neoepitope-reactive NKT cells indicates a late-phase immune response.

28. The method of any one of claims 23-27, further comprising a step of enriching the blood sample to isolate PBMC cells prior to exposing the blood sample to at least one neoepitope.

29. The method of any one of claims 23-28, wherein the blood sample is obtained a plurality of times by a predetermined time interval, and the response metric represents a change of the immune response of the patient.

30. The method of any one of claims 23-29, wherein the immune-stimulating conditions involve exposing the blood sample at least one of a lipopolysaccharide and a bacterial endotoxin.

31. The method of any one of claims 23-30, wherein the immune-stimulating conditions involve exposing the blood sample to one or more cytokines.

32. Use of patient blood sample to quantify an immune response of the patient by exposing the blood sample to at least one neoepitope under immune-stimulating conditions, quantifying the reaction of immune competent cells in the blood sample to the at least one neoepitope, characterizing at least one of the immune competent cells (a) with respect to a proportion in whole blood and/or (b) with respect to immune competent cell subtypes, respectively, to generate a response metric, wherein the immune competent cells are at least one of B cells, T cells, NK cells, and NKT cells; wherein a greater proportion of neoepitope-reactive B cells indicates an activated immune response, wherein the presence of neoepitope-reactive T cells, NK cells or NKT cells indicates a late-phase immune response, and determining to exclude or include a neoepitope-based treatment from treatment options based on the response metric.

33. The use of claim 32, wherein the immune competent cells are B cells, and characterizing at least one of the immune competent cells comprises characterizing isolated B cells (a) with respect to a proportion in whole blood and/or (b) with respect to B cell subtype, to generate a response metric, wherein a greater proportion of neoepitope-reactive B cells indicates an activated immune response.

34. The use of any one of claims 32-33, wherein the immune competent cells are T cells, and characterizing at least one of the immune competent cells comprises characterizing isolated T cells (a) with respect to a proportion in whole blood and/or (b) with respect to T cell subtype, to generate a response metric, wherein the presence of neoepitope-reactive T cells indicates a late-phase immune response.

35. The use of any one of claims 32-34, wherein the immune competent cells are NK cells, and characterizing at least one of the immune competent cells comprises characterizing isolated NK cells (a) with respect to a proportion in whole blood and/or (b) with respect to NK cell type, to generate a response metric, wherein the presence of neoepitope-reactive NK cells indicates a late-phase immune response.

36. The use of any one of claims 32-35, wherein the immune competent cells are NKT cells, and characterizing at least one of the immune competent cells comprises characterizing isolated NKT cells (a) with respect to a proportion in whole blood and/or (b) with respect to NKT cell subtype, to generate a response metric, wherein the presence of neoepitope-reactive NKT cells indicates a late-phase immune response.

37. The use of any one of claims 32-36, further comprising enriching the blood sample to isolate PBMC cells prior to exposing the blood sample to at least one neoepitope.

38. The use of any one of claims 32-37, wherein the blood sample is obtained a plurality of times by a predetermined time interval, and the response metric represents a change of the immune response of the patient.

39. The use of any one of claims 32-38, wherein the immune-stimulating conditions involve exposing the blood sample to a lipopolysaccharide and/or a bacterial endotoxin.

40. The use of any one of claims 32-39, wherein the immune-stimulating conditions involve exposing the blood sample to one or more cytokines.
